(19) United States
(12) Patent Application Publication
(10) Pub. No.: US 2024/0331806 A1
(43) Pub. Date: Oct. 3, 2024

(54) Machine Learning Based Genomics Test Predictor

(71) Applicant: Oracle International Corporation, Redwood Shores, CA (US)

(72) Inventor: Chia-Chu Sun DORLAND, Folsom, CA (US)

(21) Appl. No.: 18/192,896

(22) Filed: Mar. 30, 2023

(51) Int. Cl. G16B 40/20

(52) U.S. Cl. CPC G16B 40/20

(57) ABSTRACT

Embodiments predict genomic testing using machine learning. Embodiments receive one or more training datasets of a genomic pipeline comprising a plurality of training variables for each of a plurality of genomic tests and corresponding results of each of the genomic tests. Embodiments train a machine learning model using the training datasets and receive a new genomic workflow pipeline comprising new genomic testing variables. Embodiments then predict, using the trained machine learning model and new genomic testing variables, whether the new genomic workflow pipeline will be successfully completed within a first compute environment.

### FIELD

One embodiment is directed generally to a computer system, and in particular to genomics testing using a computer system.

### BACKGROUND INFORMATION

Whole genome sequencing (“WGS”), also known as full genome sequencing, complete genome sequencing, or entire genome sequencing, is the process of determining the entirety, or nearly the entirety, of the Deoxyribonucleic acid (“DNA”) sequence of an organism's genome at a single time. In principle, full genome sequencing can provide the raw nucleotide sequence of an individual organism's DNA at a single point in time. However, further analysis must be performed to provide the biological or medical meaning of this sequence, such as how this knowledge can be used to help prevent disease.

Genomics sequencing typically generates a large amount of data (e.g., there are approximately six billion base pairs in each human diploid genome). Its output is stored electronically and requires a large amount of computing power and storage capacity. Various companies, such as Illumina, Inc., Applied Biosystems, Oxford Nanopore Technologies, etc., have developed DNA sequencers for performing the sequencing.

### SUMMARY

Embodiments predict genomic testing using machine learning. Embodiments receive one or more training datasets of a genomic pipeline comprising a plurality of training variables for each of a plurality of genomic tests and corresponding results of each of the genomic tests. Embodiments train a machine learning model using the training datasets and receive a new genomic workflow pipeline comprising new genomic testing variables. Embodiments then predict, using the trained machine learning model and new genomic testing variables, whether the new genomic workflow pipeline will be successfully completed within a first compute environment.

### BRIEF DESCRIPTION OF THE DRAWINGS

FIG. 1 illustrates an example of a system that includes a genomics test predictor system in accordance to embodiments.

FIG. 2 is a block diagram of a genomics test predictor system of FIG. 1 in the form of a computer server/system in accordance with an embodiment of the present invention.

FIG. 3 is a block diagram of the functionality of the genomics test predictor system in accordance to an embodiment of the invention.

FIG. 4 illustrates an example dataset that may be provided to a dataset collector and can be used to train the ML model.

FIG. 5 illustrates a UI for selecting input variables as input data for the trained prediction model and displaying the test prediction in accordance with embodiments.

FIG. 6 illustrates a UI for selecting input variables as input data for the trained prediction model and displaying the test prediction in accordance with embodiments.

FIG. 7 illustrates a UI illustrating input test training dataset and sliders for selecting input variables as input data for the trained prediction model in accordance with embodiments.

FIG. 8 illustrates some example pre-configured cloud environments that are tailored for genomics pipeline execution in accordance to embodiments.

FIG. 9 is a block diagram of a GenoOKE system that can implement the selectable OKE platforms for genomic testing in accordance to embodiments.

FIG. 10 is a flow diagram of the functionality of the genomics test predictor module of FIG. 2 when predicting whether genomic tests will be successfully run based on the compute environment in accordance with one embodiment.

FIGS. 11-14 illustrate an example cloud infrastructure that can incorporate the genomic test predictor system of FIG. 1 as well as the GenoOKE system of FIG. 9 in accordance to embodiments.

Further embodiments, details, advantages, and modifications will become apparent from the following detailed description of the embodiments, which is to be taken in conjunction with the accompanying drawings.

### DETAILED DESCRIPTION

One embodiment is a machine learning based system that predicts whether one or more genomic sequencing test pipelines, each of which includes a number of genomic tests and genomic batches, can be successfully completed in a given genomics runtime compute infrastructure. Embodiments provide prompt and near immediate feedback on whether the infrastructure capacity setup supports the various number of genomics tests, instead of taking hours or days to run tests to get the results. Embodiments further provide a user interface to allow the prediction to be performed on any combination of user input ranges without wasting time and effort to manually prepare a different large number of sample tests.

Genomic sequencing pipelines refers to the series of steps and processes involved in obtaining, processing, and analyzing DNA sequence data from a biological sample. The typical steps in a genomic sequencing pipeline include: (1) Sample preparation: Obtaining a sample of DNA from a biological source, which may require extraction, purification, and quantification; (2) Library preparation: Generating a collection of DNA fragments that are suitable for sequencing, usually involving amplification and fragmentation; (3) Sequencing: Determining the exact order of nucleotides (A, C, G, T) in the DNA fragments, using a variety of sequencing technologies such as Illumina, PacBio, or Oxford Nanopore; (4) Data processing: Cleaning and transforming the raw sequencing data into a format that can be analyzed, such as demultiplexing and adapter trimming; and (5) Data analysis: Analyzing the processed data to generate useful biological insights, such as gene expression levels, variant calling, or functional annotation.

These steps are highly automated and typically use specialized software and algorithms to ensure the accuracy and efficiency of the sequencing pipeline. Software tools, such as “NetFlow”, are used for building bioinformatics pipelines. Nextflow is designed to make it easier to develop, test, and run complex genomic analysis workflows. Nextflow allows a user to write pipelines in a high-level, expressive language and then automatically handles the parallel execution and data management aspects of the analysis. This makes it easier to scale the pipelines to multiple nodes, either on a high-performance computing cluster or in the cloud. Additionally, Nextflow provides features such as checkpointing, restartability, and versioning, which make it easier to develop and maintain large, complex pipelines. Embodiments work for all pipelines management applications, such as Nextflow, Common Workflow Language (“CWL”), etc.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

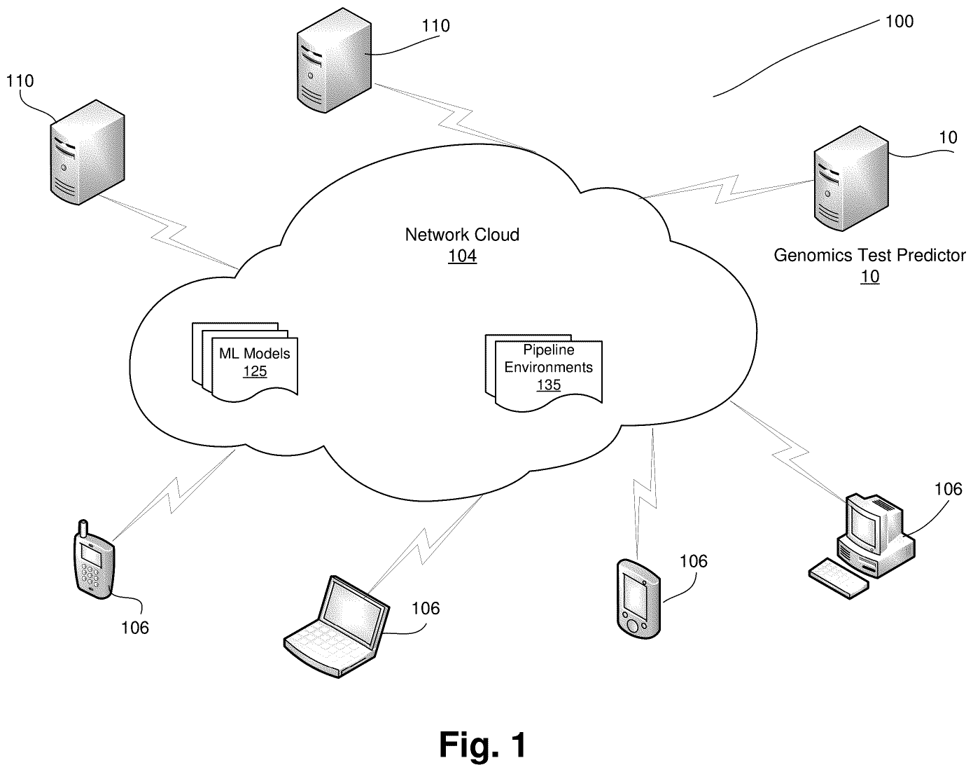

FIG. 1 illustrates an example of a system 100 that includes a genomics test predictor system 10 in accordance to embodiments. Genomics test predictor system 10 may be implemented within a computing environment that includes a communication network/cloud 104. Network 104 may be a private network that can communicate with a public network (e.g., the Internet) to access additional services 110 provided by a cloud services provider. Examples of communication networks include a mobile network, a wireless network, a cellular network, a local area network (“LAN”), a wide area network (“WAN”), other wireless communication networks, or combinations of these and other networks. Genomics test predictor system 10 may be administered as a stand-alone service, or by a service provider, such as via the Oracle Cloud Infrastructure (“OCI”) from Oracle Corp., on-premise, or a combination of cloud and on-premise.

Tenants of the cloud services provider can be organizations or groups whose members include users of services offered by service provider. Services may include or be provided as access to, without limitation, an application, a resource, a file, a document, data, media, or combinations thereof. Users may have individual accounts with the service provider and organizations may have enterprise accounts with the service provider, where an enterprise account encompasses or aggregates a number of individual user accounts.

System 100 further includes client devices 106, which can be any type of device that can access network 104 and can obtain the benefits of the functionality of Genomics test predictor system 10 of predicting the success or failure of running genomic sequencing tests. As disclosed herein, a “client” (also disclosed as a “client system” or a “client device”) may be a device or an application executing on a device. System 100 includes a number of different types of client devices 106 that each is able to communicate with network 104.

Executing on cloud 104 are one or more ML models 125. Each ML model 125 can be executed by a customer of cloud 104. In embodiments, an ML model 125 can be accessible to a client 106 via a representational state transfer application programming interface (“REST API”) and function as an endpoint to the API. ML models 125 can by any type of machine learning model that, in general, is trained on some training data and validation data and then can process additional incoming “live” data to make predictions. Examples of ML models 125 include artificial neural networks (“ANN”), decision trees, support-vector machines (“SVM”), Bayesian networks, etc. Training data can be any set of data capable of training ML model 125 (e.g., a set of features with corresponding labels, such as labeled data for supervised learning). In embodiments, training data can be used to train a ML model 125 to generate a trained ML model 125.

Further executing on cloud 104 are one or more pipeline computing environments 135. Each pipeline environment 135 can be a preconfigured computing environment having a predefined amount of resources (e.g., processing power, memory, etc.) configured to execute genomic pipelines. In one embodiment, each pipeline environment is containerized in the form of a container such as “Docker” or “Kubernetes” and is managed by Kubernetes. In embodiments where cloud 104 is implemented with the OCI, each pipeline environment 135 is implemented as a Oracle Cloud Infrastructure Container Engine for Kubernetes (“OKE”), which is a fully-managed, scalable, and highly available service used to deploy containerized applications (i.e., genomics pipelines) to the cloud. A user can specify the compute resources that the application requires, and OKE provisions them on OCI in an existing OCI tenancy.

For example, a Nextflow pipeline has multiple tasks for analyzing Illumina samples. These tasks can run as containerized program. When these containerized tasks are deployed to a Kubernetes cluster environment, Kubernetes will manage these containers (e.g., dockers) via a Pod form. Kubernetes in general manages Pods which contains dockers (i.e., containerized programs). OKE is OCI's Kubernetes reference implementation.

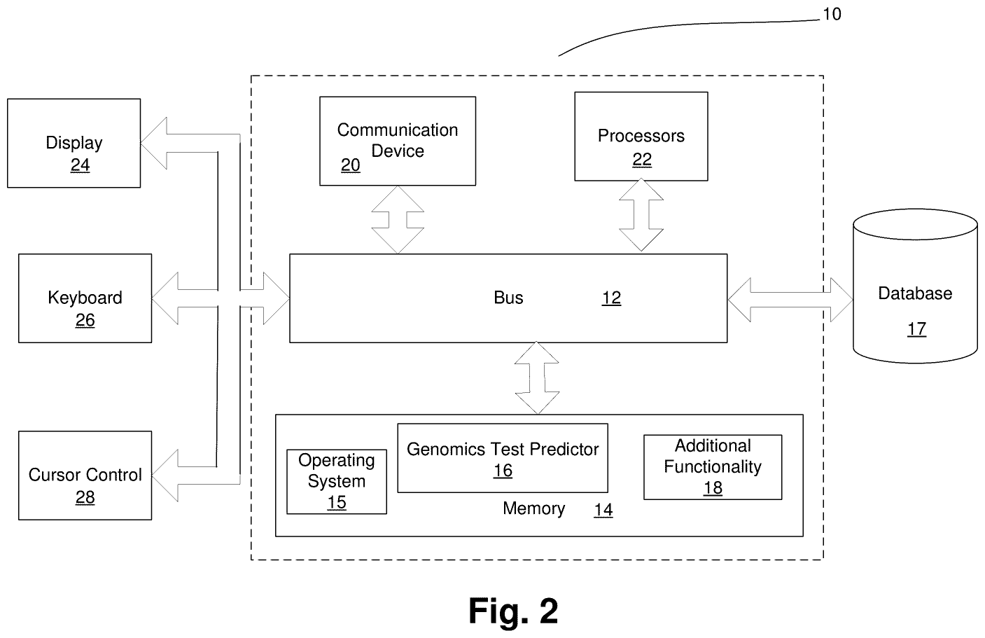

FIG. 2 is a block diagram of a genomics test predictor system 10 of FIG. 1 in the form of a computer server/system 10 in accordance with an embodiment of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. One or more components of FIG. 2 can also be used to implement any of the elements of FIG. 1 and FIGS. 3 and 9, discussed below.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication device 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a genomics test predictor module 16 that predicts whether genomic tests will be successful, and all other functionality disclosed herein. System 10 can be part of a larger system. Therefore, system 10 can include one or more additional functional modules 18 to include the additional functionality, such as any other functionality provided by the Oracle Cloud Infrastructure (“OCI”) from Oracle Corp. A file storage device or database 17 is coupled to bus 12 to provide centralized storage for modules 16 and 18, including data regarding previous schema mappings. In one embodiment, database 17 is a relational database management system (“RDBMS”) that can use Structured Query Language (“SQL”) to manage the stored data.

As discussed, the computing infrastructure resources and capacity for the genomics runtime environment, regardless of hosted in cloud or non-cloud, is often under software, hardware and cost constraints. Genomics computation, as its nature, can involve enormous amounts of data and require unpredictable compute resources and capacity. Scientist users often need to prepare and run different number and type stress tests to verify the infrastructure runtime capacity. However, even with the stress tests, the genomic tests may ultimately fail, meaning all tests in the pipelines do not finish, after a long running time (e.g., 2-3 days). This wastes time and effort.

In general, the tests may fail because of infrastructure resource constraints on CPU/memory/storage/compute type. Genomic analysis pipelines often have different compute resource requirements depending on the data that it needs to process. When a large number of pipelines is being executed concurrently, the infrastructure resources are being shared. The pipelines that require large resources for their run times and could not get the required compute resources within a certain time would fail. A failed pipeline could impact other pipelines because of dependencies and cause other pipelines to fail.

In contrast, embodiments can predict in milliseconds whether the resources deployed in the genomics runtime environment have the capacity to support all prepared sample tests managed by the workflow system (e.g., Nextflow) to run completely and successfully. Genomics sample sequencing and analysis could take up hours and days. Embodiments train and deploy a machine learning based on a collected training dataset and predicts the test results on the fly based on the user input. Embodiments not only help genomics test preparation but also improves resource management in the genomics runtime infrastructure.

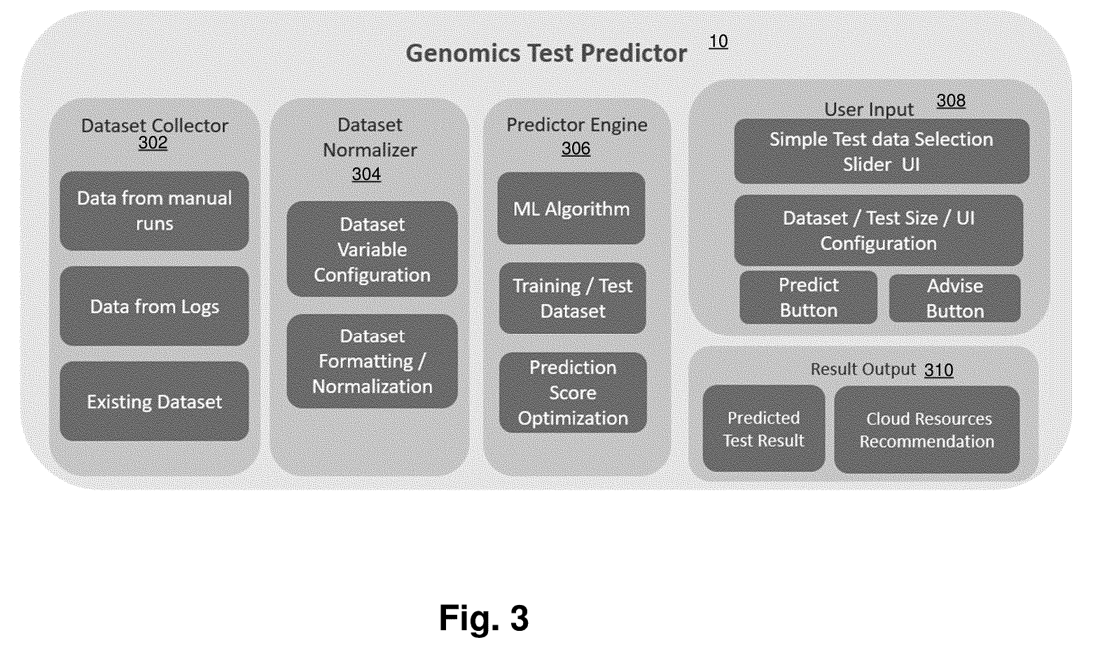

FIG. 3 is a block diagram of the functionality of genomics test predictor system 10 in accordance to an embodiment of the invention.

System 10 includes a dataset collector 302 that is used to train an ML model. The prediction dataset initially can be collected through manual runs or can be copied from a previous dataset, or can be automatically collected over time through logs parsing. The number of features in embodiments should be a minimum of five and no greater than twelve. All the features in embodiments will be automatically converted to sliders displayed in a UI as disclosed below. In embodiments with greater than twelve features, only twelve of the features may be converted to sliders. The UI will provide an option to let user change the feature label. For example, users can label “Illumina Samples” to “IllumSam”, and then a normalized comma-separated values (“csv”) file and slider label will reflect that.

The raw data can in the form of a csv file that was entered manually by a test engineer, or it can be a dataset that already has the valid format and was used previously by system 10. In general, it is very common to generate log files when running programs or stress tests for troubleshooting. For example, a stress test log file may look like the following, from which system 10 would parse the log file to get the data it needs to generate the dataset:
2023-02-16 12:00:40 INFO Starting stress test . . .
2023-02-16 12:00:40 INFO Running 100 Illumina tests, each test will be run 50 times
2023-02-16 12:00:40 INFO Test 1: Running Illumina test 1 of 100 (iteration 1 of 50)
. . .
2023-02-16 12:00:40 INFO Test 100: Illumina test 100 of 100 (iteration 50 of 50) completed successfully

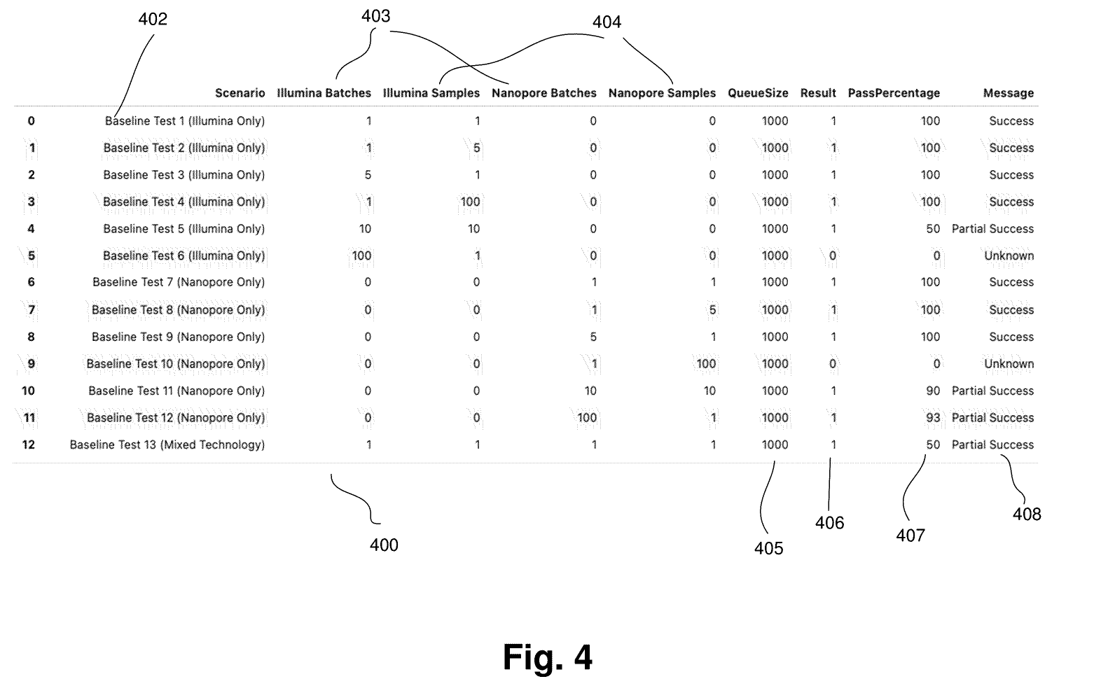

FIG. 4 illustrates an example dataset that may be provided to dataset collector 302 and can be used to train the ML model. Dataset 400 includes a listing of genomic sample tests scenarios in column 402. Each row of FIG. 4 shows a Nextflow run which was managing different pipelines for the sequencing analysis work for Illumina or Nanopore or both. FIG. 4 does not illustrate an entire pipeline, but instead shows different genomics sequencing work that was executed via the Nextflow workflow/pipeline orchestration. FIG. 4 is an example of what a raw dataset could look like, and is a partial csv example file containing each stress test result plus extra columns for any message and comments. The raw data can be collected by a test engineer manually and entered as a csv file, or automatically generated.

Each pipeline includes the number of each type of test batches (e.g., Illumina batches and Nanopore batches) at 403 and type of sample tests (e.g., Illumina samples and Nanopore samples) at 404. For example, run Illumina 10 batches on Illumina 5 samples means to execute an Illumina test on 5 sequencing samples divided into 10 batches.

FIG. 4 further illustrates a queue size for the respective pipeline at 405, the result of the tests at 406, which is normalized to a “1” or “0” (“1” is success or partial success, “0” is unknown). “Unknown” in the raw csv file indicates that the stress test did not return any test results as “Success” or “Failed”, or “OutOfResource” or “Partially Success.”

FIG. 4 further illustrates a pass percentage at 407, and a message regarding the outcome of the tests at 408. Test predictor 10 is adapted to output a result of the tests (i.e., as in column 406) in response to live input data after the ML model is trained. Because the compute infrastructure that the stress tests are running in are fixed in embodiments (i.e., the compute resources are constant) the actual compute environment is not needed for the prediction (which assumes the same computer environment will be used). However, if the compute resources vary for each scenario, then the compute resources (e.g., CPU/memory) can be added to the dataset.

Although the embodiment shown in FIG. 4 is implemented with Nextflow, other embodiments can use different workflow management systems, such as CWL. In embodiments, workflow management systems configuration can be added as an independent variable via the UI. For example, as shown in FIG. 4, “QueueSize” is one of the independent variables (i.e., feature) for Nextflow pipelines configuration. For CWL pipelines, the similar configuration is called “MaxConcurrent”. If the raw dataset received by system 10 does not contain any data for ‘QueueSize’ or ‘MaxConcurrent’ configuration, the generated dataset base will not contain the corresponding column.

System 10 further includes a dataset normalizer 304 that parses the input data, removes constant variables or text messages, converts variable values to numeric values, and fills in missing values. FIG. 4 is a raw dataset example. The generated normalized dataset in embodiments will not contain Scenario, Message and PassPercentage.

User input 308 allows a user to provide configuration options to specify additional base data variables that were collected in the raw data to be used by dataset normalizer 304. For example, the user can add the workflow program queue size, or the number of processor or nodes that were allocated for test runs. If this configuration data are available in the raw dataset and users also specify this data are features, when system 10 generates the normalized dataset, it will include this data as part of the final dataset.

User input 308 further provides configuration options to specify the test size, to modify the normalized dataset values, or to add additional data columns. User input 308, in embodiments, implements simple and intuitive UI components to show data labels to allow easy input of the number of batches, tests, type of samples and workflow variables for the prediction. For example, sliders, disclosed below, will be automatically generated for each header/feature of the input dataset in the UI. Users have options to configure which data headers should be displayed in the UI. User input 308 further includes a “predict” button to initiate a prediction, and an “advise” button. Based on the predicted results, if the result is “Failed to Complete” and the compute resources, CPU/memory is part of the dataset, the “advise” button will use the data as the baseline to advise the appropriate total number of CPU/memory or other compute/cloud resources should be used.

System 10 further includes a predictor engine 306 that includes the ML model/algorithm, which can be implemented by one or more of ML models 125. The ML model is trained using the input data from 302, such as the simplified dataset shown in FIG. 4. After being trained, live data is input to generate an prediction output. In one embodiment, the ML model is a supervised ML Logistic Regression algorithm/model. However, the ML model can also be implemented. Predictor engine 306 is adapted to predict in milliseconds whether the resources deployed in the existing genomics runtime environment (e.g., on-premise or cloud based resources) has the capacity to support all prepared genomics sample tests managed by the workflow system to completely run successfully.

System 10 further includes a result output 310 that includes a “Predict” button to immediately generate predicted results based on the user input. Result output 310, in embodiments, can further provide cloud resource recommendations for running the tests, based on the resource needs of the tests.

In one embodiment, system 10 is integrated with an artificial intelligence (“Al”) based chatbot such as the “ChatGPT” from “OpenAI”. The integration uses the chatbot to not only support general prompts, but also can provide the test predictor service to the users based on their specified dataset. In embodiments that use the ChatGPT, a WebUI link of “https://genormics.test.predictor.ai” will be available for users to access. The UI includes the test predictor UI components (e.g., configurations, sliders, buttons, output area), a prompt entry and a prompt response display area. In the UI backend, the genomics GPT integrator is test predictor plus OpenAI-GPT. It adds a specific genomics test predictor data model or trained data to the GPT3's text-davinci-003 model to provide more information on specific genomics prompts that are not available from current general prompts.

A test predictor prompts builder and parser handles user prompts and identifies whether the prompt is a general prompt or specifically a prompt for the genomics test predictor. For example, for genomics specific prompts, such as specifying the raw dataset location, prompts can include what the independent variables or features are for the genomics test predictor, a request for a more detailed explanation on the returned test results, or a request for recommendations on cloud resources and their costs. In response, the test predictor prompts builder and parser will detect all these genomics specific prompts and will forward them to a test predictor GPT Response Actor for proper actions. A completion generator will generate the complete response by combining test predictor results and information found in GPT.

In one embodiment, all prompts started with/OTP or/Oracle Test Predictor will be immediately answered or actioned in the most recent OTP context. For example:
Ex0: /OTP: show me an example of how to use OTP.
Ex1: /OTP: raw dataset A-raw-data-file is located in http://github/my-data, the independent features are A-seq-sample, B-seq-sample, A-batch, B-batch, CPU. show me the generated dataset after OTP normalized it.
Ex2: /OTP: why would the stress test fail if I ran A-seq-sample 50 samples with 10 batches using total 32 cpus?

Integrating with Chat-GPT can help users understand the prediction results by explaining them in an intuitive manner. It can also answer any questions the users might have about the raw data collected, the normalized dataset and the prediction results. Embodiments that are integrated with an Al based chatbot provide users immediately with the ability to interact with system 10 in connection with all functionality used to generate and provide the prediction. It also provide the users with a quick and easier way to access different genomics data, analysis tools and cloud resource information, and at the same time some proprietary data that is pertinent to system 10 only.

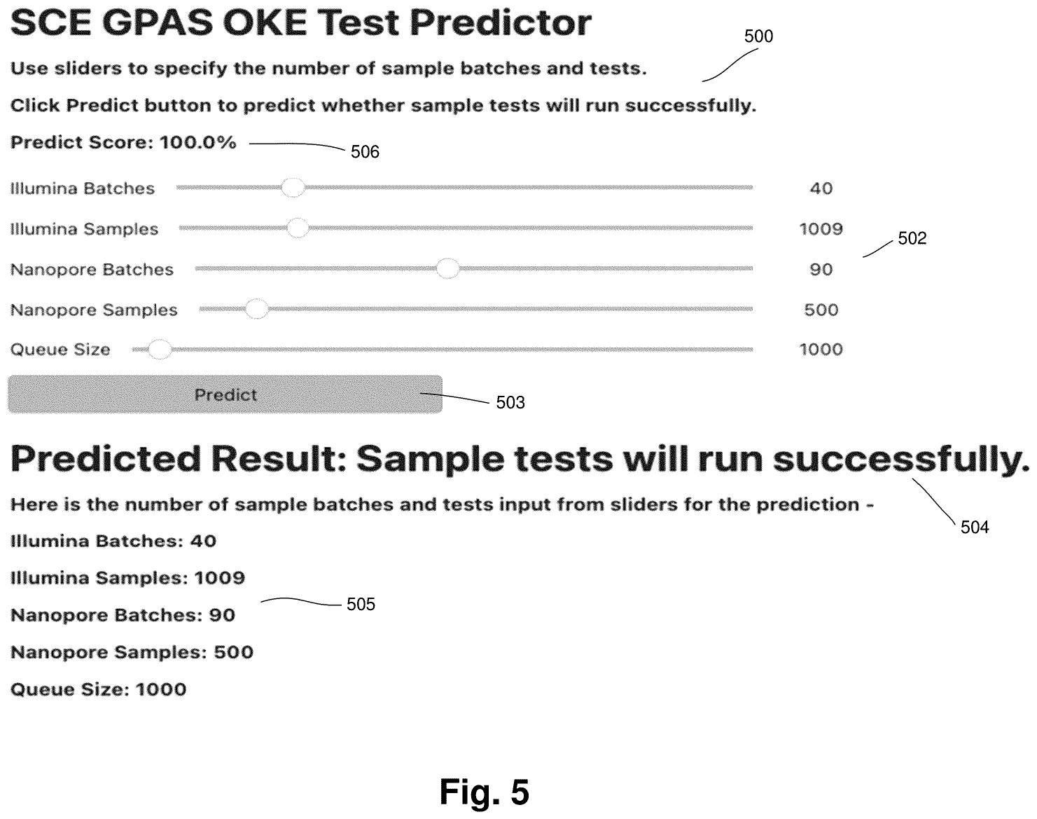

FIG. 5 illustrates a UI 500 for selecting input variables as input data for the trained prediction model and displaying the test prediction in accordance with embodiments. The variables 502, which correspond to the independent variables from the training dataset that was used to train the ML model, are displayed in the form of a group of sliders, so a user can easily revise the value of each variable. The use then selects the “predict” button 503, and the predicted results are generated and displayed at 504 and 505. Further, a test prediction score may be displayed at 506. The prediction scores is generated by the ML model when the predict button is clicked. It is used internally as reference for optimization. In embodiments, system 10 will change the parameter values a few times in the linear regression model (e.g., LogisticRegress(C=[10, 50, 90],solver=[Ibfgs,saga, liblinear], max_iter[200,300,400]) to find the most optimized test score. When a relatively simple ML LogisticRegression model is used, optimizing the test prediction score is fairly quick.

As disclosed, if the compute environment is constant or fixed, the type of compute environment will have no impact on the test results. In this case, similar to the provided raw data, the different number of stress tests had been running in the same environment, so that the test results depend on the number of sample types, the number of samples and the number of the batches. If the stress tests had been run in different compute environments, then computer environment variables such as CPU/memory/others could be added as part of the dataset.

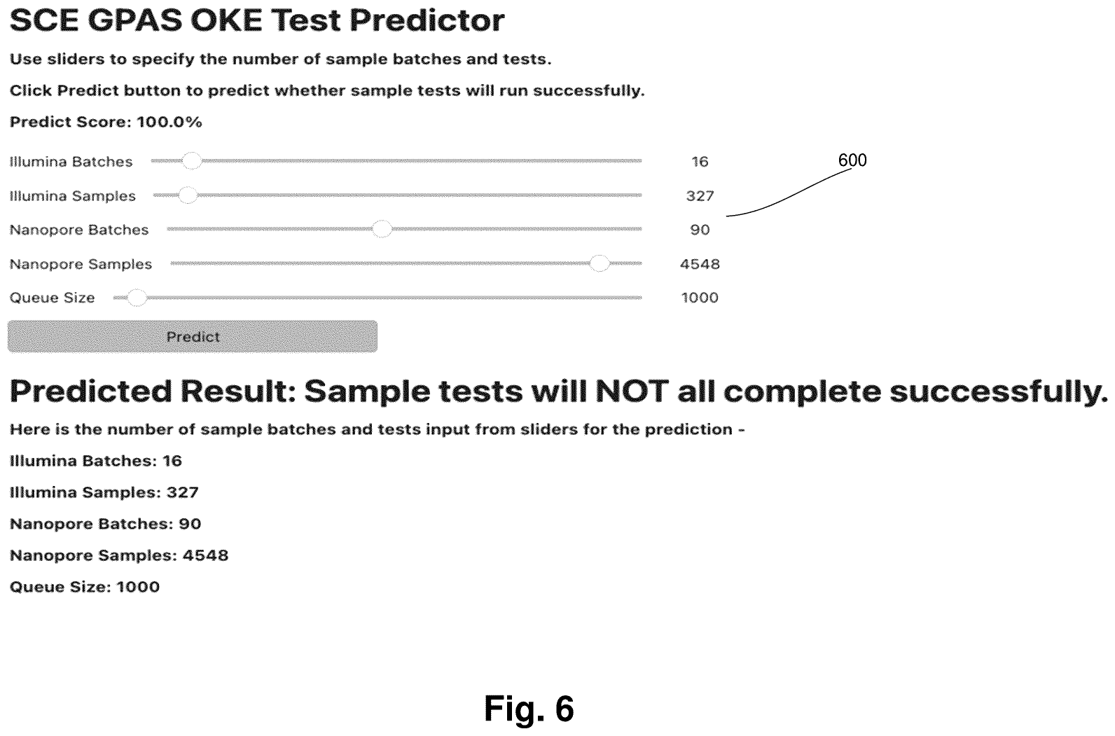

FIG. 6 illustrates a UI 600 for selecting input variables as input data for the trained prediction model and displaying the test prediction in accordance with embodiments. In contrasts to the tests in FIG. 5, in FIG. 6 the result of the prediction is the sample tests will not all complete successfully.

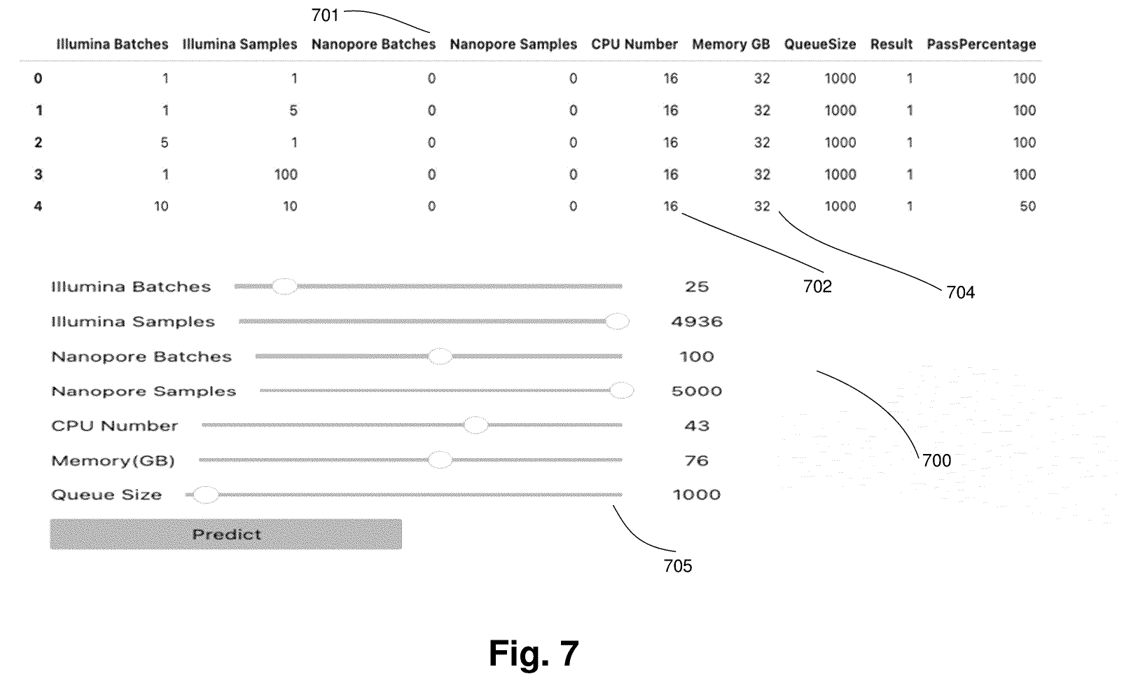

FIG. 7 illustrates a UI 700 illustrating input test training dataset and sliders for selecting input variables as input data for the trained prediction model in accordance with embodiments. The test dataset 700 is similar to the test dataset of FIG. 4 but includes two extra variables: the number of CPUs 702 and amount of memory 704 that was used to implement each pipeline. Sliders 705 include the additional variables. In embodiments, variables related to the compute environment such as variables 702 and 704 are not used to predict whether the tests will run successfully, but instead are used to predict or recommend a compute environment for running the tests. In one embodiment, a plurality of pre-configured cloud environments will be available, and the recommended environment will be automatically selected.

In embodiments, if features such as variables 702 and 704 in FIG. 7 are constant numbers, they will not influence the prediction so they will not be shown as sliders. However, if they have different row values, then they can contribute to the prediction. Users can adjust the 702/704 values along with other features to see what is the capacity they need for their compute system. The testPredictor system 10 will, based on the test results and the cpu/memory used, recommend the proper out-of-box GenoOKE suite.

Once a decision has been made to run a pipeline, in response to a prediction by system 10 that the test will run successfully, embodiments can select an appropriate compute environment based on resources required, costs, etc. As disclosed, in embodiments additional independent variables can be used in the test training dataset and live input data to generate test prediction. In one embodiment, additional variables relate to the computing environment that can run the tests. With known solutions, if Nextflow users would like to run pipelines in Kubernetes hosted in cloud, they are required to have some infrastructure knowledge or have someone to assist in creating the Kubernetes cluster in the cloud, in addition to the effort to configure the cluster for running Nextflow pipelines. However, the time and overhead needed to create and set up a Kubernetes cluster to make it ready to run Nextflow pipelines are much longer and larger than merely creating batches of compute instances.

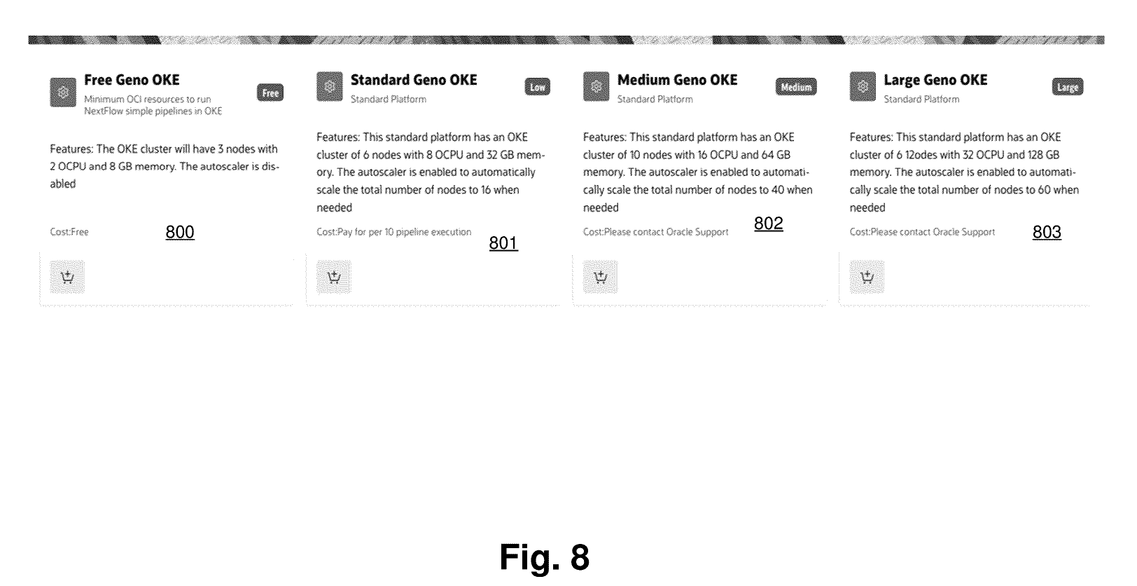

In contrast, embodiments provide a list of pre-built Kubernetes cluster environments with OCI resources, or other cloud resources, or on-premise resources that are tailored specifically for Nextflow pipeline execution. FIG. 8 illustrates some example pre-configured cloud environments that are tailored for genomics pipeline execution in accordance to embodiments. In one embodiment, each of 800-803 is a pre-built OCI environment that is tailored for the Nextflow Pipeline execution and can be manually selectable, or automatically selectable based on the output of test predictor system 10. Each out-of-box genomics platform 800-803 in embodiments includes varied OCI resources such as an OKE cluster, file storage service (“FSS”), Object Storage and Nextflow required images and setups. In other embodiments that implement a cloud service other than OCI or on-premises, different resources may be configured.

Embodiments implement a “GenoOKE” service in conjunction with test prediction system 10 that provides a list of available out-of-box and ready-to-run genomics OKE platforms, such as platforms/environments 800-803. GenoOKE is an all-inclusive genomics cloud based service. It provides needed compute resources, storage, networking and tools for genomics pipeline executions. It has fixed price so recourse usage does not matter. Users just need to grab, pay and run the genome pipelines in it.

The GenoOKE platform includes varied OCI resources, such as OKE cluster for Nextflow Kubernetes runs, compute instances for bastion and admin hosts, FSS for Nextflow Kubernetes pods storage claim, OCIR for containerized images and Object Storage for unlimited storage. The Nextflow users can select one of the available running GenoOKE platforms, agree to the cost to receive all needed access information and ready to run their Nextflow pipelines, or a platform is automatically selected by test prediction system 10.

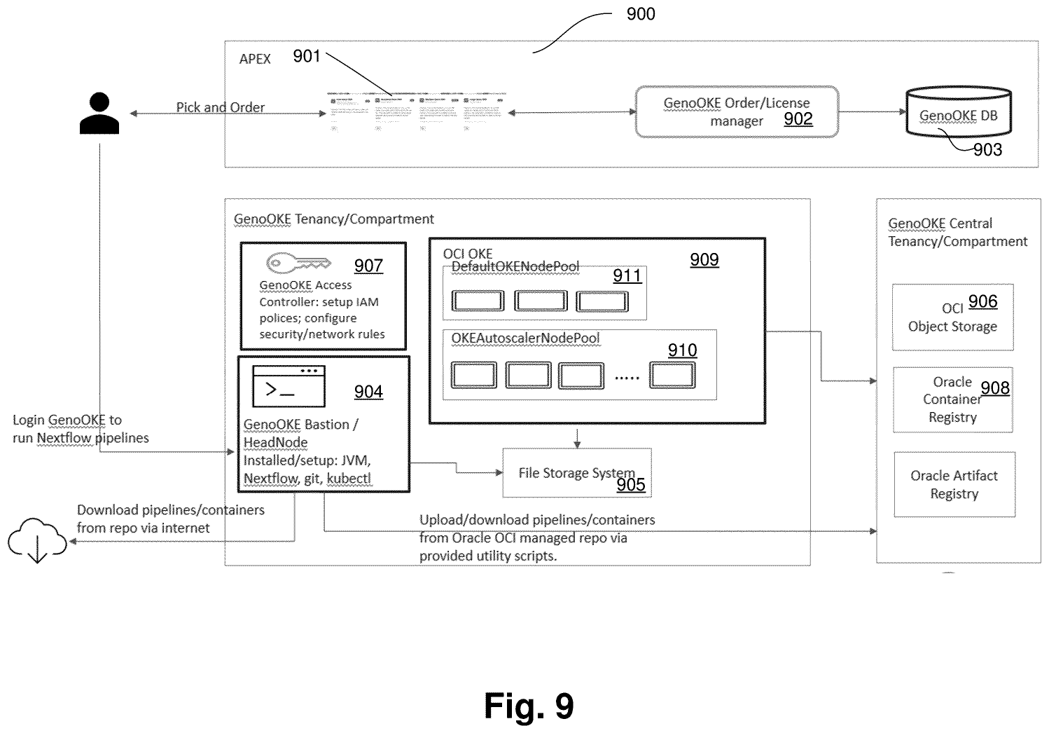

FIG. 9 is a block diagram of a GenoOKE system 900 that can implement the selectable OKE platforms for genomic testing in accordance to embodiments. Any or all portions of GenoOKE system 900 can be implemented by system 10.

The GenoOKE will have various editions 901, such as Free GenoOKE 800, Standard GenoOKE 801, Medium GenoOKE 802 and Large GenoOKE 803. The differences between different GenoOKE editions generally focus on the total number of CPUs and memory of instances that are available for a genomics pipelines run. In embodiments, all GenoOKE editions have the same basic modules and OCI resources as shown in FIG. 9. They are pre-built and configured like a software appliance specifically for running Nextflow pipelines. The cost models can vary for different editions. It could be free trial, pay per day, pay by sample runs or pay by license.

A GenoOKE Order/License manager 902 handles the ordering and payment models. A GenoOKE DB 903 stores all ordering data and GenoOKE HeadNode 904 login information.

Each edition 901 after build will automatically create a user/admin with password and paired Secure Shell Protocol (“ssh”) keys for accessing GenoOKE Headnode 904. It will also create required OCI policies for HeadNode 904 to access OKE, FSS 905 and Object Storage 906. Users will be able to ssh to Headnode 904 and run Nextflow pipelines.

After the user GenoOKE order, the GenoOKE Order/License Manager will provide the following login info:

User id: <genooke-headnode-user-name>
User password: <genooke-headnode-user-pwd>
Ssh private key: <genooke-user-ssh-private-key>
Ssh public keyL<genooke-headnode-ssh-public-key>
Headnode public ip: <genooke-headnode-public-ip-address>
Headnode domain name:<genooke-headnode-host-name>

Users will be able to ssh to the GenoOKE Headnode and run Nextflow pipelines.

For the standard GenoOKE 801, in embodiments, a GenoOKE access controller 907 will set up OCI IAM required users/instance/service policy and configure security and network rules. It will also generate a HeadNode user/password, paired ssh keys and store the login information in GenoOKE DB 903.

GenoOKE HeadNode 904 is a Linux based instance and built on top of OCI flexible shapes (e.g., VM.Standard3.Flex) and Oracle Linux images. This instance is pre-installed with JVM, Nextflow, python, git, Kubernetes client application and other software required to run Nextflow. It is also configured with NFS mounts to access FSS 905 and Object Storage 906 directly. It allows downloading container images or pipelines from the Internet. It also has utility scripts to upload, download or list container images from Oracle Container Registry (“OCIR”) 908.

OKE 909, for the standard GenoOKE 801, is a Kubernetes containing 2 cluster node pools 911. Autoscaling is enabled. One of them will be used by the Cluster Autoscaler 910. The shapes of the work nodes of the node pool will be OCI flexible shapes with Oracle Linux images. Each cluster node instance has 16 CPUs and 32 GB memory. There will be minimum default total number of 6 cluster nodes but can be scaled up to max number of 16 nodes.

FSS 905 will be configured and presented as exports as “/data” and “/work” and shared by GenoOKE HeadNode 904 and all work nodes of the OKE cluster.

Object Storage 906 will be created for any type of data storage. It can be accessed from HeadNode 904, the OCI Console, or via an API with token. When the lease is expired, the data/logs/content stored in Object Storage 906 will be available for a year.

OCIR 908 is pre-configured to access OCIR public containers. A private OCIR repo will be created. An OCIR utility tool will be provided to list available container images.

For the free GenoOKE 800, in embodiments, instances (nodes) created will have limited CPUs and memory. For example, HeadNode 904 will have 2 CPUs and 1 GB. Only 1 node pool of 3 nodes are included for the OKE cluster 911. Cluster Autoscaler 910 is disabled. FSS has limitations. Once the Free trial period is expired, all OCI resources including Object Storage 906 will be deleted.

For the medium GenoOKE 802, in embodiments, the shapes of the work nodes of the node pool will also be OCI flexible shapes with Oracle Linux images but with larger number of CPUs and memory than the Standard edition 801. For example, each cluster node instance has 32 CPUs and 64 GB memory. There will be a minimum default total number of 10 cluster nodes. Since the OKE Cluster Autoscaler 910 is enabled, this edition is able to automatically scale the total number of nodes from the minimum 10 nodes to 40 nodes when needed. When the lease is expired, the data/logs/content stored in Object Storage 906 will be available for 2 years.

For the large GenoOKE 803, in embodiments, this edition is similar to the Medium edition 802 but it is able to automatically scale the total number of nodes from minimum 12 nodes to 60 nodes when needed. The node instances will have a larger number of CPUs and memory. For example, each node will have 64 CPUs and 128 GB. When the lease is expired, the data/logs/content stored in Object Storage 906 will be available for unlimited years.

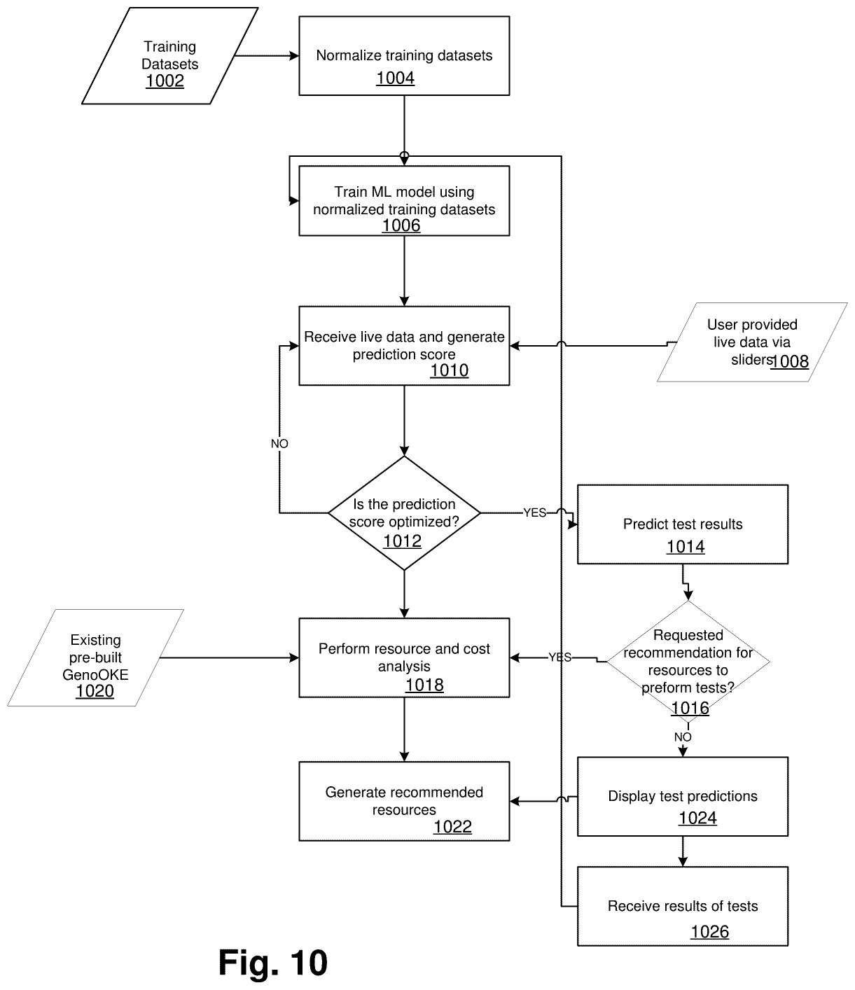

FIG. 10 is a flow diagram of the functionality of genomics test predictor module 16 of FIG. 2 when predicting whether genomic tests will be successfully run based on the compute environment in accordance with one embodiment. In one embodiment, the functionality of the flow diagram of FIG. 10 is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

In general, the test predictor system 10 in the functionality of FIG. 10 predicts whether existing computing resources have the capacity to support all prepared genomic pipeline sample tests that can be successfully completed. If the prediction indicates the existing environment will not be sufficient for the planned genomics pipeline analysis to run, it will provide the optimal recommendations on what pre-built cloud resources are available and will be sufficient to run the pipelines along with information on their costs.

At 1002, training datasets are retrieved. The training datasets initially can be collected through manual runs or can be copied from a previous dataset, or can be automatically collected over time through logs parsing

At 1004, the training datasets are normalized. For example, the results of the tests are normalized to a “1” or “0”.

At 1006, the ML model is trained using the normalized training datasets from 1004.

At 1008, the “live” input data 1009 is received from the user. The live data includes variables that match one or more variables used as the training dataset. The live data can be selected/generated by the user using sliders on a UI that are generated in response to the training dataset.

At 1010, the trained ML model calculates a prediction test score in response to receiving the live data.

At 1012, it is determined if the prediction test score is the most optimized score, such as being greater than a predefined minimum (e.g., 0.6). For example, the parameters will be changed 10 times (or other predefined number) with different values on the ML method call and the best score is chosen for the prediction.

If no at 1012 (e.g., the prediction score is not greater than 0.6), additional data will be requested for training the model and the functionality will continue at 1010.

If yes at 1012, at 1014 the result of the test prediction is determined.

At 1016, it is determined if the user requested recommendations for pre-built cloud Kubernetes resources (e.g., pre-built GenoOKE 800-803) or recommendations for a suggested environments.

For example, users can adjust the CPU/memory (702/704 of FIG. 7) values to predict their desired number of genomics features to determine whether the existing compute environment has the capacity to complete the genomics sample tests. When test predictor system 10 predicts the number of sample tests with the number of CPU/memory will not all complete successfully, it will recommend the next upgraded compute shapes. Users can also prompt the integrated Chat-GPT for different compute upgrade shapes.

If no at 1016, at 1024 the test prediction results are displayed. If yes at 1016, at 1018 cloud genomics resource and cost analyzer receives the existing pre-built resources at 1020 and then at 1022 generates recommended resources, which may be a recommendation of one of the pre-built resources, or suggestions for a new set of resources, and can include costs. The costs are based on the suggested compute infrastructure (e.g., CPU, memory, other cloud resources) and the corresponding known costs of those resources. The results are displayed at 1024.

For example, for OCI, there is a set of pre-configured OCI based GenoOKE and users are looking for an upgraded and hands-off runtime environment for their genomics analysis pipelines. The Chat-GPT integrated TestPredict will answer “/OTP:” prompts with a list of all-inclusive pre-configured GenoOKE editions and needed details.

At 1026, after the tests are run, likely successfully as predicted, the results of the tests are used to retrain the model at 1012.

### Example Cloud Infrastructure

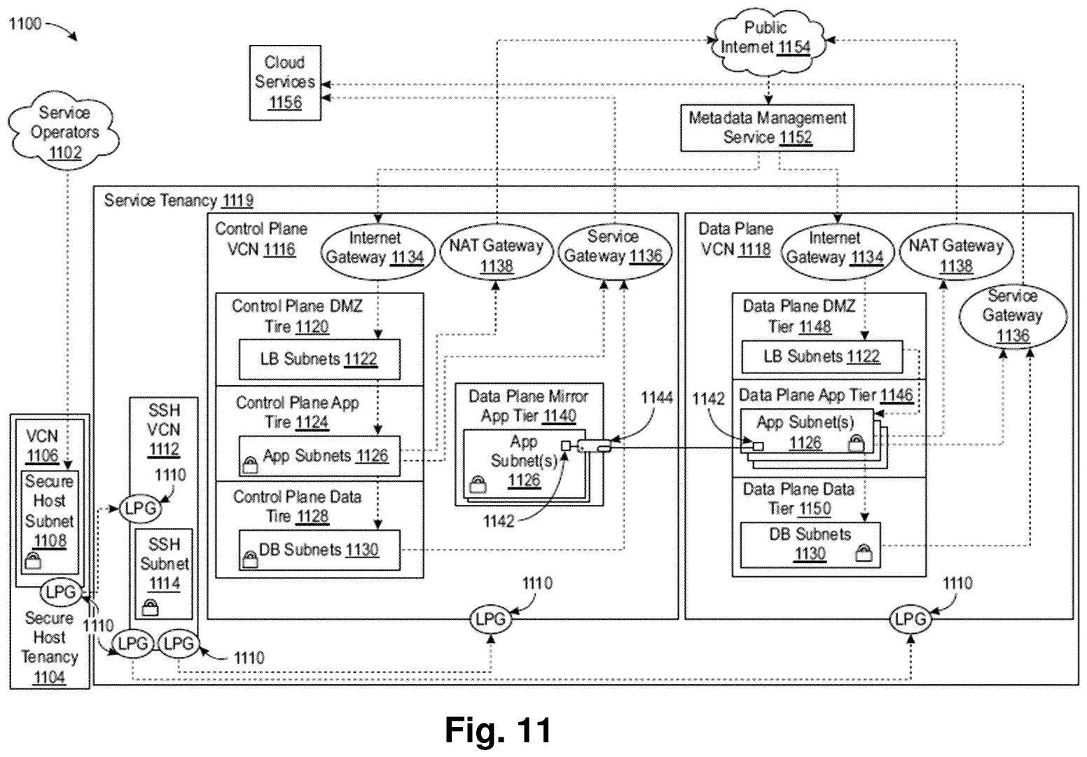

FIGS. 11-14 illustrate an example cloud infrastructure that can incorporate the genomic test predictor system 10 of FIG. 1 as well as the GenoOKE system 900 of FIG. 9 in accordance to embodiments.

As disclosed above, infrastructure as a service (“IaaS”) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (e.g., billing, monitoring, logging, security, load balancing and clustering, etc.). Thus, as these services may be policy-driven, IaaS users may be able to implement policies to drive load balancing to maintain application availability and performance.

In some instances, IaaS customers may access resources and services through a wide area network (“WAN”), such as the Internet, and can use the cloud provider's services to install the remaining elements of an application stack. For example, the user can log in to the IaaS platform to create virtual machines (“VM”s), install operating systems (“OS”s) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different problems for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (“VPC”s) (e.g., a potentially on-demand pool of configurable and/or shared computing resources), also known as a core network. In some examples, there may also be one or more security group rules provisioned to define how the security of the network will be set up and one or more virtual machines. Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 11 is a block diagram 1100 illustrating an example pattern of an IaaS architecture, according to at least one embodiment. Service operators 1102 can be communicatively coupled to a secure host tenancy 1104 that can include a virtual cloud network (“VCN”) 1106 and a secure host subnet 1108. In some examples, the service operators 1102 may be using one or more client computing devices, which may be portable handheld devices (e.g., an iPhone®, cellular telephone, an iPad®, computing tablet, a personal digital assistant (“PDA”)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (“SMS”), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1106 and/or the Internet.

The VCN 1106 can include a local peering gateway (“LPG”) 1110 that can be communicatively coupled to a secure shell (“SSH”) VCN 1112 via an LPG 1110 contained in the SSH VCN 1112. The SSH VCN 1112 can include an SSH subnet 1114, and the SSH VCN 1112 can be communicatively coupled to a control plane VCN 1116 via the LPG 1110 contained in the control plane VCN 1116. Also, the SSH VCN 1112 can be communicatively coupled to a data plane VCN 1118 via an LPG 1110. The control plane VCN 1116 and the data plane VCN 1118 can be contained in a service tenancy 1119 that can be owned and/or operated by the IaaS provider.

The control plane VCN 1116 can include a control plane demilitarized zone (“DMZ”) tier 1120 that acts as a perimeter network (e.g., portions of a corporate network between the corporate intranet and external networks). The DMZ-based servers may have restricted responsibilities and help keep security breaches contained. Additionally, the DMZ tier 1120 can include one or more load balancer (“LB”) subnet(s) 1122, a control plane app tier 1124 that can include app subnet(s) 1126, a control plane data tier 1128 that can include database (DB) subnet(s) 1130 (e.g., frontend DB subnet(s) and/or backend DB subnet(s)). The LB subnet(s) 1122 contained in the control plane DMZ tier 1120 can be communicatively coupled to the app subnet(s) 1126 contained in the control plane app tier 1124 and an Internet gateway 1134 that can be contained in the control plane VCN 1116, and the app subnet(s) 1126 can be communicatively coupled to the DB subnet(s) 1130 contained in the control plane data tier 1128 and a service gateway 1136 and a network address translation (NAT) gateway 1138. The control plane VCN 1116 can include the service gateway 1136 and the NAT gateway 1138.

The control plane VCN 1116 can include a data plane mirror app tier 1140 that can include app subnet(s) 1126. The app subnet(s) 1126 contained in the data plane mirror app tier 1140 can include a virtual network interface controller (VNIC) 1142 that can execute a compute instance 1144. The compute instance 1144 can communicatively couple the app subnet(s) 1126 of the data plane mirror app tier 1140 to app subnet(s) 1126 that can be contained in a data plane app tier 1146.

The data plane VCN 1118 can include the data plane app tier 1146, a data plane DMZ tier 1148, and a data plane data tier 1150. The data plane DMZ tier 1148 can include LB subnet(s) 1122 that can be communicatively coupled to the app subnet(s) 1126 of the data plane app tier 1146 and the Internet gateway 1134 of the data plane VCN 1118. The app subnet(s) 1126 can be communicatively coupled to the service gateway 1136 of the data plane VCN 1118 and the NAT gateway 1138 of the data plane VCN 1118. The data plane data tier 1150 can also include the DB subnet(s) 1130 that can be communicatively coupled to the app subnet(s) 1126 of the data plane app tier 1146.

The Internet gateway 1134 of the control plane VCN 1116 and of the data plane VCN 1118 can be communicatively coupled to a metadata management service 1152 that can be communicatively coupled to public Internet 1154. Public Internet 1154 can be communicatively coupled to the NAT gateway 1138 of the control plane VCN 1116 and of the data plane VCN 1118. The service gateway 1136 of the control plane VCN 1116 and of the data plane VCN 1118 can be communicatively coupled to cloud services 1156.

In some examples, the service gateway 1136 of the control plane VCN 1116 or of the data plane VCN 1118 can make application programming interface (“API”) calls to cloud services 1156 without going through public Internet 1154. The API calls to cloud services 1156 from the service gateway 1136 can be one-way: the service gateway 1136 can make API calls to cloud services 1156, and cloud services 1156 can send requested data to the service gateway 1136. But, cloud services 1156 may not initiate API calls to the service gateway 1136.

In some examples, the secure host tenancy 1104 can be directly connected to the service tenancy 1119, which may be otherwise isolated. The secure host subnet 1108 can communicate with the SSH subnet 1114 through an LPG 1110 that may enable two-way communication over an otherwise isolated system. Connecting the secure host subnet 1108 to the SSH subnet 1114 may give the secure host subnet 1108 access to other entities within the service tenancy 1119.

The control plane VCN 1116 may allow users of the service tenancy 1119 to set up or otherwise provision desired resources. Desired resources provisioned in the control plane VCN 1116 may be deployed or otherwise used in the data plane VCN 1118. In some examples, the control plane VCN 1116 can be isolated from the data plane VCN 1118, and the data plane mirror app tier 1140 of the control plane VCN 1116 can communicate with the data plane app tier 1146 of the data plane VCN 1118 via VNICs 1142 that can be contained in the data plane mirror app tier 1140 and the data plane app tier 1146.

In some examples, users of the system, or customers, can make requests, for example create, read, update, or delete (“CRUD”) operations, through public Internet 1154 that can communicate the requests to the metadata management service 1152. The metadata management service 1152 can communicate the request to the control plane VCN 1116 through the Internet gateway 1134. The request can be received by the LB subnet(s) 1122 contained in the control plane DMZ tier 1120. The LB subnet(s) 1122 may determine that the request is valid, and in response to this determination, the LB subnet(s) 1122 can transmit the request to app subnet(s) 1126 contained in the control plane app tier 1124. If the request is validated and requires a call to public Internet 1154, the call to public Internet 1154 may be transmitted to the NAT gateway 1138 that can make the call to public Internet 1154. Memory that may be desired to be stored by the request can be stored in the DB subnet(s) 1130.

In some examples, the data plane mirror app tier 1140 can facilitate direct communication between the control plane VCN 1116 and the data plane VCN 1118. For example, changes, updates, or other suitable modifications to configuration may be desired to be applied to the resources contained in the data plane VCN 1118. Via a VNIC 1142, the control plane VCN 1116 can directly communicate with, and can thereby execute the changes, updates, or other suitable modifications to configuration to, resources contained in the data plane VCN 1118.

In some embodiments, the control plane VCN 1116 and the data plane VCN 1118 can be contained in the service tenancy 1119. In this case, the user, or the customer, of the system may not own or operate either the control plane VCN 1116 or the data plane VCN 1118. Instead, the IaaS provider may own or operate the control plane VCN 1116 and the data plane VCN 1118, both of which may be contained in the service tenancy 1119. This embodiment can enable isolation of networks that may prevent users or customers from interacting with other users', or other customers', resources. Also, this embodiment may allow users or customers of the system to store databases privately without needing to rely on public Internet 1154, which may not have a desired level of security, for storage.

In other embodiments, the LB subnet(s) 1122 contained in the control plane VCN 1116 can be configured to receive a signal from the service gateway 1136. In this embodiment, the control plane VCN 1116 and the data plane VCN 1118 may be configured to be called by a customer of the IaaS provider without calling public Internet 1154. Customers of the IaaS provider may desire this embodiment since database(s) that the customers use may be controlled by the IaaS provider and may be stored on the service tenancy 1119, which may be isolated from public Internet 1154.

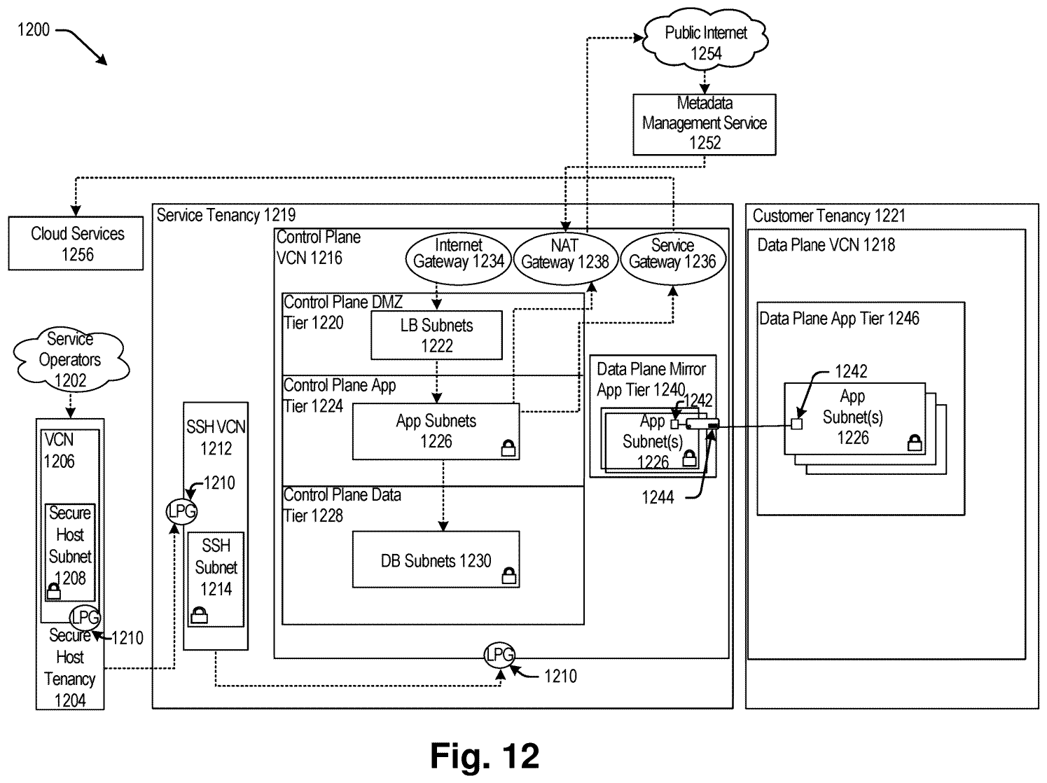

FIG. 12 is a block diagram 1200 illustrating another example pattern of an IaaS architecture, according to at least one embodiment. Service operators 1202 (e.g. service operators 1102) can be communicatively coupled to a secure host tenancy 1204 (e.g. the secure host tenancy 1104) that can include a virtual cloud network (VCN) 1206 (e.g. the VCN 1106) and a secure host subnet 1208 (e.g. the secure host subnet 1108). The VCN 1206 can include a local peering gateway (LPG) 1210 (e.g. the LPG 1110) that can be communicatively coupled to a secure shell (SSH) VCN 1212 (e.g. the SSH VCN 1112 10) via an LPG 1110 contained in the SSH VCN 1212. The SSH VCN 1212 can include an SSH subnet 1214 (e.g. the SSH subnet 1114), and the SSH VCN 1212 can be communicatively coupled to a control plane VCN 1216 (e.g. the control plane VCN 1116) via an LPG 1210 contained in the control plane VCN 1216. The control plane VCN 1216 can be contained in a service tenancy 1219 (e.g. the service tenancy 1119), and the data plane VCN 1218 (e.g. the data plane VCN 1118) can be contained in a customer tenancy 1221 that may be owned or operated by users, or customers, of the system.

The control plane VCN 1216 can include a control plane DMZ tier 1220 (e.g. the control plane DMZ tier 1120) that can include LB subnet(s) 1222 (e.g. LB subnet(s) 1122), a control plane app tier 1224 (e.g. the control plane app tier 1124) that can include app subnet(s) 1226 (e.g. app subnet(s) 1126), a control plane data tier 1228 (e.g. the control plane data tier 1128) that can include database (DB) subnet(s) 1230 (e.g. similar to DB subnet(s) 1130). The LB subnet(s) 1222 contained in the control plane DMZ tier 1220 can be communicatively coupled to the app subnet(s) 1226 contained in the control plane app tier 1224 and an Internet gateway 1234 (e.g. the Internet gateway 1134) that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 1238 (e.g. the NAT gateway 1138). The control plane VCN 1216 can include the service gateway 1236 and the NAT gateway 1238.

The control plane VCN 1216 can include a data plane mirror app tier 1240 (e.g. the data plane mirror app tier 1140) that can include app subnet(s) 1226. The app subnet(s) 1226 contained in the data plane mirror app tier 1240 can include a virtual network interface controller (VNIC) 1242 (e.g. the VNIC of 1142) that can execute a compute instance 1244 (e.g. similar to the compute instance 1144). The compute instance 1244 can facilitate communication between the app subnet(s) 1226 of the data plane mirror app tier 1240 and the app subnet(s) 1226 that can be contained in a data plane app tier 1246 (e.g. the data plane app tier 1146) via the VNIC 1242 contained in the data plane mirror app tier 1240 and the VNIC 1242 contained in the data plane app tier 1246.

The Internet gateway 1234 contained in the control plane VCN 1216 can be communicatively coupled to a metadata management service 1252 (e.g. the metadata management service 1152) that can be communicatively coupled to public Internet 1254 (e.g. public Internet 1154). Public Internet 1254 can be communicatively coupled to the NAT gateway 1238 contained in the control plane VCN 1216. The service gateway 1236 contained in the control plane VCN 1216 can be communicatively couple to cloud services 1256 (e.g. cloud services 1156).

In some examples, the data plane VCN 1218 can be contained in the customer tenancy 1221. In this case, the IaaS provider may provide the control plane VCN 1216 for each customer, and the IaaS provider may, for each customer, set up a unique compute instance 1244 that is contained in the service tenancy 1219. Each compute instance 1244 may allow communication between the control plane VCN 1216, contained in the service tenancy 1219, and the data plane VCN 1218 that is contained in the customer tenancy 1221. The compute instance 1244 may allow resources that are provisioned in the control plane VCN 1216 that is contained in the service tenancy 1219, to be deployed or otherwise used in the data plane VCN 1218 that is contained in the customer tenancy 1221.

In other examples, the customer of the IaaS provider may have databases that live in the customer tenancy 1221. In this example, the control plane VCN 1216 can include the data plane mirror app tier 1240 that can include app subnet(s) 1226. The data plane mirror app tier 1240 can reside in the data plane VCN 1218, but the data plane mirror app tier 1240 may not live in the data plane VCN 1218. That is, the data plane mirror app tier 1240 may have access to the customer tenancy 1221, but the data plane mirror app tier 1240 may not exist in the data plane VCN 1218 or be owned or operated by the customer of the IaaS provider. The data plane mirror app tier 1240 may be configured to make calls to the data plane VCN 1218, but may not be configured to make calls to any entity contained in the control plane VCN 1216. The customer may desire to deploy or otherwise use resources in the data plane VCN 1218 that are provisioned in the control plane VCN 1216, and the data plane mirror app tier 1240 can facilitate the desired deployment, or other usage of resources, of the customer.

In some embodiments, the customer of the IaaS provider can apply filters to the data plane VCN 1218. In this embodiment, the customer can determine what the data plane VCN 1218 can access, and the customer may restrict access to public Internet 1254 from the data plane VCN 1218. The IaaS provider may not be able to apply filters or otherwise control access of the data plane VCN 1218 to any outside networks or databases. Applying filters and controls by the customer onto the data plane VCN 1218, contained in the customer tenancy 1221, can help isolate the data plane VCN 1218 from other customers and from public Internet 1254.

In some embodiments, cloud services 1256 can be called by the service gateway 1236 to access services that may not exist on public Internet 1254, on the control plane VCN 1216, or on the data plane VCN 1218. The connection between cloud services 1256 and the control plane VCN 1216 or the data plane VCN 1218 may not be live or continuous. Cloud services 1256 may exist on a different network owned or operated by the IaaS provider. Cloud services 1256 may be configured to receive calls from the service gateway 1236 and may be configured to not receive calls from public Internet 1254. Some cloud services 1256 may be isolated from other cloud services 1256, and the control plane VCN 1216 may be isolated from cloud services 1256 that may not be in the same region as the control plane VCN 1216. For example, the control plane VCN 1216 may be located in “Region 1,” and cloud service “Deployment 8,” may be located in Region 1 and in “Region 2.” If a call to Deployment 8 is made by the service gateway 1236 contained in the control plane VCN 1216 located in Region 1, the call may be transmitted to Deployment 8 in Region 1. In this example, the control plane VCN 1216, or Deployment 8 in Region 1, may not be communicatively coupled to, or otherwise in communication with, Deployment 8 in Region 2.

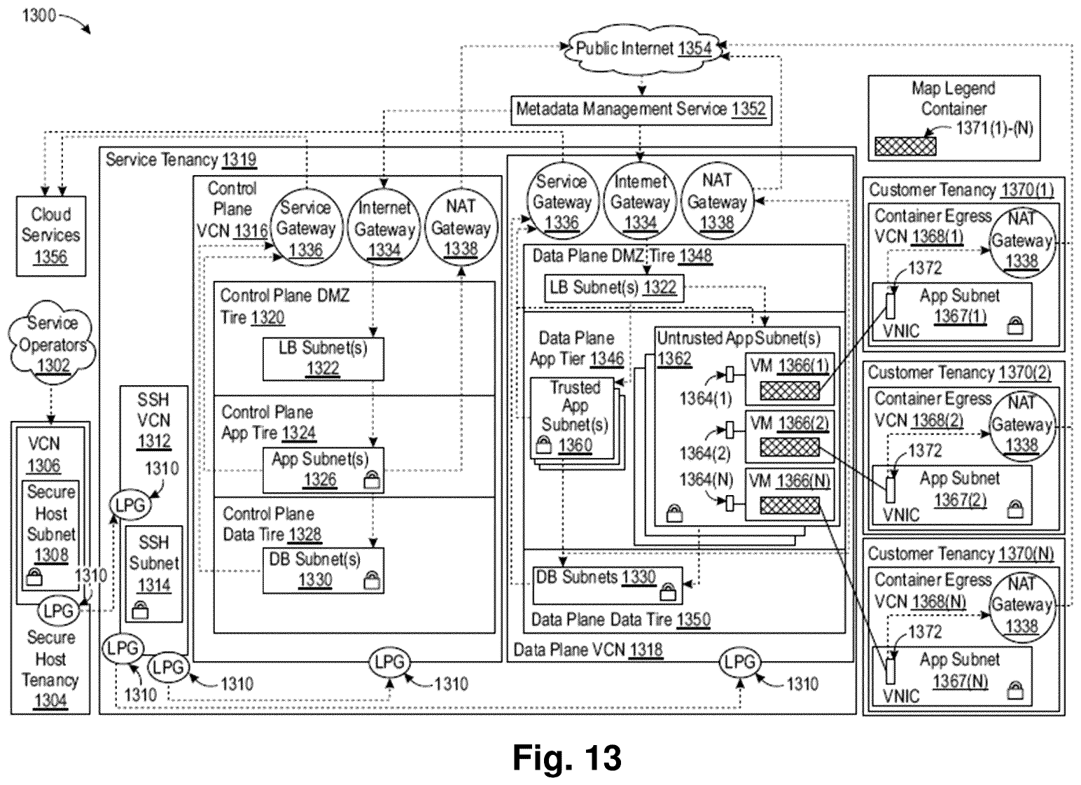

FIG. 13 is a block diagram 1300 illustrating another example pattern of an IaaS architecture, according to at least one embodiment. Service operators 1302 (e.g. service operators 1102) can be communicatively coupled to a secure host tenancy 1304 (e.g. the secure host tenancy 1104) that can include a virtual cloud network (VCN) 1306 (e.g. the VCN 1106) and a secure host subnet 1308 (e.g. the secure host subnet 1108). The VCN 1306 can include an LPG 1310 (e.g. the LPG 1110) that can be communicatively coupled to an SSH VCN 1312 (e.g. the SSH VCN 1112) via an LPG 1310 contained in the SSH VCN 1312. The SSH VCN 1312 can include an SSH subnet 1314 (e.g. the SSH subnet 1114), and the SSH VCN 1312 can be communicatively coupled to a control plane VCN 1316 (e.g. the control plane VCN 1116) via an LPG 1310 contained in the control plane VCN 1316 and to a data plane VCN 1318 (e.g. the data plane 1118) via an LPG 1310 contained in the data plane VCN 1318. The control plane VCN 1316 and the data plane VCN 1318 can be contained in a service tenancy 1319 (e.g. the service tenancy 1119).

The control plane VCN 1316 can include a control plane DMZ tier 1320 (e.g. the control plane DMZ tier 1120) that can include load balancer (“LB”) subnet(s) 1322 (e.g. LB subnet(s) 1122), a control plane app tier 1324 (e.g. the control plane app tier 1124) that can include app subnet(s) 1326 (e.g. similar to app subnet(s) 1126), a control plane data tier 1328 (e.g. the control plane data tier 1128) that can include DB subnet(s) 1330. The LB subnet(s) 1322 contained in the control plane DMZ tier 1320 can be communicatively coupled to the app subnet(s) 1326 contained in the control plane app tier 1324 and to an Internet gateway 1334 (e.g. the Internet gateway 1134) that can be contained in the control plane VCN 1316, and the app subnet(s) 1326 can be communicatively coupled to the DB subnet(s) 1330 contained in the control plane data tier 1328 and to a service gateway 1336 (e.g. the service gateway) and a network address translation (NAT) gateway 1338 (e.g. the NAT gateway 1138). The control plane VCN 1316 can include the service gateway 1336 and the NAT gateway 1338.

The data plane VCN 1318 can include a data plane app tier 1346 (e.g. the data plane app tier 1146), a data plane DMZ tier 1348 (e.g. the data plane DMZ tier 1148), and a data plane data tier 1350 (e.g. the data plane data tier 1150 of FIG. 11). The data plane DMZ tier 1348 can include LB subnet(s) 1322 that can be communicatively coupled to trusted app subnet(s) 1360 and untrusted app subnet(s) 1362 of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include one or more primary VNICs 1364(1)-(N) that can be communicatively coupled to tenant virtual machines (VMs) 1366(1)-(N). Each tenant VM 1366(1)-(N) can be communicatively coupled to a respective app subnet 1367(1)-(N) that can be contained in respective container egress VCNs 1368(1)-(N) that can be contained in respective customer tenancies 1370(1)-(N). Respective secondary VNICs 1372(1)-(N) can facilitate communication between the untrusted app subnet(s) 1362 contained in the data plane VCN 1318 and the app subnet contained in the container egress VCNs 1368(1)-(N). Each container egress VCNs 1368(1)-(N) can include a NAT gateway 1338 that can be communicatively coupled to public Internet 1354 (e.g. public Internet 1154).

The Internet gateway 1334 contained in the control plane VCN 1316 and contained in the data plane VCN 1318 can be communicatively coupled to a metadata management service 1352 (e.g. the metadata management system 1152) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively couple to cloud services 1356.

In some embodiments, the data plane VCN 1318 can be integrated with customer tenancies 1370. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane tier app 1346. Code to run the function may be executed in the VMs 1366(1)-(N), and the code may not be configured to run anywhere else on the data plane VCN 1318. Each VM 1366(1)-(N) may be connected to one customer tenancy 1370. Respective containers 1371(1)-(N) contained in the VMs 1366(1)-(N) may be configured to run the code. In this case, there can be a dual isolation (e.g., the containers 1371(1)-(N) running code, where the containers 1371(1)-(N) may be contained in at least the VM 1366(1)-(N) that are contained in the untrusted app subnet(s) 1362), which may help prevent incorrect or otherwise undesirable code from damaging the network of the IaaS provider or from damaging a network of a different customer. The containers 1371(1)-(N) may be communicatively coupled to the customer tenancy 1370 and may be configured to transmit or receive data from the customer tenancy 1370. The containers 1371(1)-(N) may not be configured to transmit or receive data from any other entity in the data plane VCN 1318. Upon completion of running the code, the IaaS provider may kill or otherwise dispose of the containers 1371(1)-(N).

In some embodiments, the trusted app subnet(s) 1360 may run code that may be owned or operated by the IaaS provider. In this embodiment, the trusted app subnet(s) 1360 may be communicatively coupled to the DB subnet(s) 1330 and be configured to execute CRUD operations in the DB subnet(s) 1330. The untrusted app subnet(s) 1362 may be communicatively coupled to the DB subnet(s) 1330, but in this embodiment, the untrusted app subnet(s) may be configured to execute read operations in the DB subnet(s) 1330. The containers 1371(1)-(N) that can be contained in the VM 1366(1)-(N) of each customer and that may run code from the customer may not be communicatively coupled with the DB subnet(s) 1330.

In other embodiments, the control plane VCN 1316 and the data plane VCN 1318 may not be directly communicatively coupled. In this embodiment, there may be no direct communication between the control plane VCN 1316 and the data plane VCN 1318. However, communication can occur indirectly through at least one method. An LPG 1310 may be established by the IaaS provider that can facilitate communication between the control plane VCN 1316 and the data plane VCN 1318. In another example, the control plane VCN 1316 or the data plane VCN 1318 can make a call to cloud services 1356 via the service gateway 1336. For example, a call to cloud services 1356 from the control plane VCN 1316 can include a request for a service that can communicate with the data plane VCN 1318.

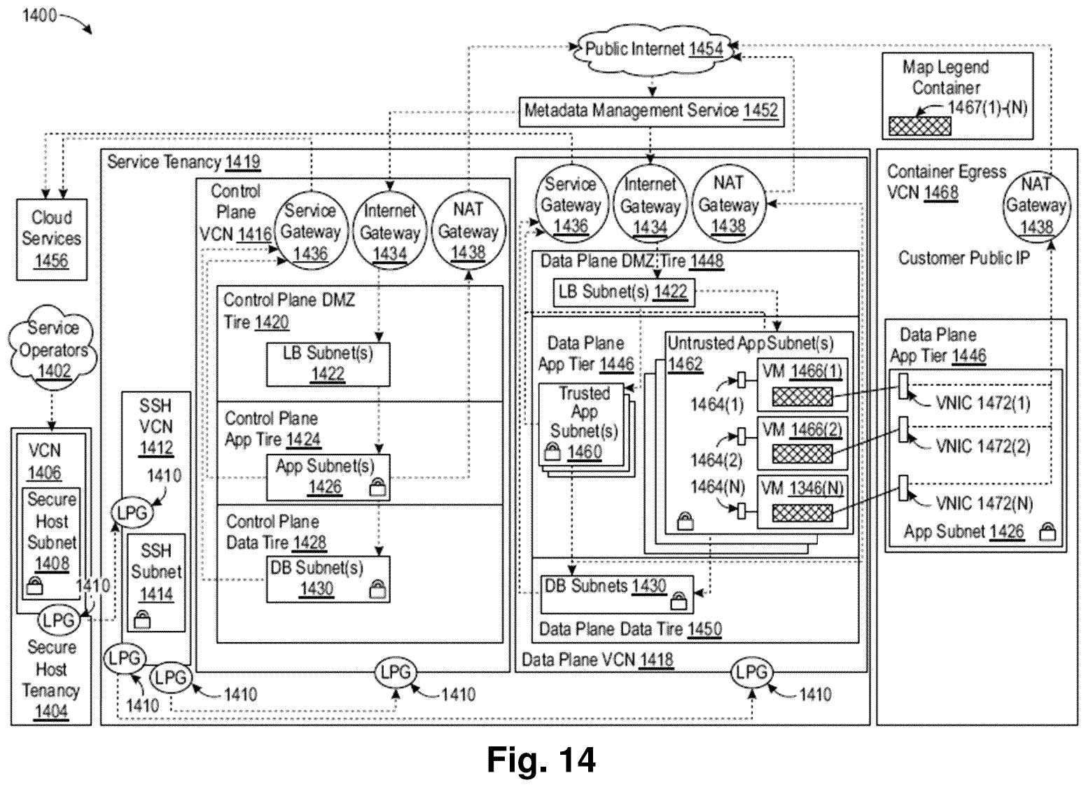

FIG. 14 is a block diagram 1400 illustrating another example pattern of an IaaS architecture, according to at least one embodiment. Service operators 1402 (e.g. service operators 1102) can be communicatively coupled to a secure host tenancy 1404 (e.g. the secure host tenancy 1104) that can include a virtual cloud network (“VCN”) 1406 (e.g. the VCN 1106) and a secure host subnet 1408 (e.g. the secure host subnet 1108). The VCN 1406 can include an LPG 1410 (e.g. the LPG 1110) that can be communicatively coupled to an SSH VCN 1412 (e.g. the SSH VCN 1112) via an LPG 1410 contained in the SSH VCN 1412. The SSH VCN 1412 can include an SSH subnet 1414 (e.g. the SSH subnet 1114), and the SSH VCN 1412 can be communicatively coupled to a control plane VCN 1416 (e.g. the control plane VCN 1116) via an LPG 1410 contained in the control plane VCN 1416 and to a data plane VCN 1418 (e.g. the data plane 1118) via an LPG 1410 contained in the data plane VCN 1418. The control plane VCN 1416 and the data plane VCN 1418 can be contained in a service tenancy 1419 (e.g. the service tenancy 1119).

The control plane VCN 1416 can include a control plane DMZ tier 1420 (e.g. the control plane DMZ tier 1120) that can include LB subnet(s) 1422 (e.g. LB subnet(s) 1122), a control plane app tier 1424 (e.g. the control plane app tier 1124) that can include app subnet(s) 1426 (e.g. app subnet(s) 1126), a control plane data tier 1428 (e.g. the control plane data tier 1128) that can include DB subnet(s) 1430 (e.g. DB subnet(s) 1330). The LB subnet(s) 1422 contained in the control plane DMZ tier 1420 can be communicatively coupled to the app subnet(s) 1426 contained in the control plane app tier 1424 and to an Internet gateway 1434 (e.g. the Internet gateway 1134) that can be contained in the control plane VCN 1416, and the app subnet(s) 1426 can be communicatively coupled to the DB subnet(s) 1430 contained in the control plane data tier 1428 and to a service gateway 1436 (e.g. the service gateway of FIG. 11) and a network address translation (NAT) gateway 1438 (e.g. the NAT gateway 1138 of FIG. 11). The control plane VCN 1416 can include the service gateway 1436 and the NAT gateway 1438.

The data plane VCN 1418 can include a data plane app tier 1446 (e.g. the data plane app tier 1146), a data plane DMZ tier 1448 (e.g. the data plane DMZ tier 1148), and a data plane data tier 1450 (e.g. the data plane data tier 1150). The data plane DMZ tier 1448 can include LB subnet(s) 1422 that can be communicatively coupled to trusted app subnet(s) 1460 (e.g. trusted app subnet(s) 1360) and untrusted app subnet(s) 1462 (e.g. untrusted app subnet(s) 1362) of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include primary VNICs 1464(1)-(N) that can be communicatively coupled to tenant virtual machines (VMs) 1466(1)-(N) residing within the untrusted app subnet(s) 1462. Each tenant VM 1466(1)-(N) can run code in a respective container 1467(1)-(N), and be communicatively coupled to an app subnet 1426 that can be contained in a data plane app tier 1446 that can be contained in a container egress VCN 1468. Respective secondary VNICs 1472(1)-(N) can facilitate communication between the untrusted app subnet(s) 1462 contained in the data plane VCN 1418 and the app subnet contained in the container egress VCN 1468. The container egress VCN can include a NAT gateway 1438 that can be communicatively coupled to public Internet 1454 (e.g. public Internet 1154).

The Internet gateway 1434 contained in the control plane VCN 1416 and contained in the data plane VCN 1418 can be communicatively coupled to a metadata management service 1452 (e.g. the metadata management system 1152) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively couple to cloud services 1456.

In some examples, the pattern illustrated by the architecture of block diagram 1400 of FIG. 14 may be considered an exception to the pattern illustrated by the architecture of block diagram 1300 of FIG. 13 and may be desirable for a customer of the IaaS provider if the IaaS provider cannot directly communicate with the customer (e.g., a disconnected region). The respective containers 1467(1)-(N) that are contained in the VMs 1466(1)-(N) for each customer can be accessed in real-time by the customer. The containers 1467(1)-(N) may be configured to make calls to respective secondary VNICs 1472(1)-(N) contained in app subnet(s) 1426 of the data plane app tier 1446 that can be contained in the container egress VCN 1468. The secondary VNICs 1472(1)-(N) can transmit the calls to the NAT gateway 1438 that may transmit the calls to public Internet 1454. In this example, the containers 1467(1)-(N) that can be accessed in real-time by the customer can be isolated from the control plane VCN 1416 and can be isolated from other entities contained in the data plane VCN 1418. The containers 1467(1)-(N) may also be isolated from resources from other customers.

In other examples, the customer can use the containers 1467(1)-(N) to call cloud services 1456. In this example, the customer may run code in the containers 1467(1)-(N) that requests a service from cloud services 1456. The containers 1467(1)-(N) can transmit this request to the secondary VNICs 1472(1)-(N) that can transmit the request to the NAT gateway that can transmit the request to public Internet 1454. Public Internet 1454 can transmit the request to LB subnet(s) 1422 contained in the control plane VCN 1416 via the Internet gateway 1434. In response to determining the request is valid, the LB subnet(s) can transmit the request to app subnet(s) 1426 that can transmit the request to cloud services 1456 via the service gateway 1436.

It should be appreciated that IaaS architectures 1100, 1200, 1300, 1400 depicted in the figures may have other components than those depicted. Further, the embodiments shown in the figures are only some examples of a cloud infrastructure system that may incorporate certain embodiments. In some other embodiments, the IaaS systems may have more or fewer components than shown in the figures, may combine two or more components, or may have a different configuration or arrangement of components.

As disclosed, embodiments provide an immediate or near immediate feedback/prediction as to whether an infrastructure capacity setup supports a number of genomic tests to be run. Embodiments further can recommend a compute environment that will run the tests, in the event the prediction that the tests will not run successfully, or an otherwise optimized compute environment based on costs, size, available preconfigured cloud environments, etc.

Embodiments convert the genomics pipeline execution elements (e.g., number of sample tests, batches, CPU and memory) in a raw dataset to normalized features and applies ML to predict whether the total number of pipelines can be completely executed within the runtime environment. The list of features can be configured and selected from the UI to be displayed as a group of sliders for users to specify the input values for each feature for the prediction.

In embodiments, thee test prediction will generally only take less than a second to predict whether the specified number of features will all complete successfully or will not all complete successfully. In addition to the test prediction, embodiments also provide an “Advise” ability for upgrading the runtime resources. For example, based on what runtime resources were being used when the tests did not all complete, it will provide recommendations on the number of CPUs, compute shapes or pre-configured genoOKE that should be used.

Embodiments can be built as a standalone App without integration with Chat-GPT or can be built as a Web Service integrated with Chat-GPT or other Al chat assistant. On the test predictor Al chat page, users will see the same TestPredictor standalone UI plus the Chat-GPT Prompts text area. Users can prefix their prompts with “/OPT:” or “/Oracle TestPredictor:” to get specific answers to their test prediction results and advised pre-configure runtime resources.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of predicting genomic testing using machine learning, the method comprising:
receiving one or more training datasets of a genomic pipeline comprising a plurality of training variables for each of a plurality of genomic tests and corresponding results of each of the genomic tests;
training a machine learning model using the training datasets;
receiving a new genomic workflow pipeline comprising new genomic testing variables; and
predicting, using the trained machine learning model and new genomic testing variables, whether the new genomic workflow pipeline will be successfully completed within a first compute environment.

2. The method of claim 1, wherein the training datasets comprise, for each of a plurality of genomic tests, a corresponding batch size, sample size and queue size.

3. The method of claim 1, wherein the machine learning model comprises a supervised logistic regression model.

4. The method of claim 1, further comprising generating a user interface with a plurality of input elements that correspond to the new genomic testing variables.

5. The method of claim 4, wherein the input elements comprising sliders.

6. The method of claim 1, wherein the new genomic testing variables correspond to the plurality of training variables.

7. The method of claim 1, wherein the training variables comprising a corresponding compute environment.

8. The method of claim 7, wherein the training variables comprises an amount of memory and a number of central processing units, the predicting further comprising a recommendation of a new compute environment for executing the new genomic workflow pipeline.

9. The method of claim 8, wherein the recommendation comprises a selection of one of a plurality of pre-configured cloud compute environments, the recommendation based at least in part on a cost of each of the pre-configured cloud compute environments.

10. The method of claim 9, further comprising providing an artificial intelligence based chatbot for responding to prompts regarding the pre-configured cloud compute environments.

11. A genomic test prediction system comprising
one or more processors executing instructions to generate a prediction, the generating the prediction comprising:
receiving one or more training datasets of a genomic pipeline comprising a plurality of training variables for each of a plurality of genomic tests and corresponding results of each of the genomic tests;
training a machine learning model using the training datasets;
receiving a new genomic workflow pipeline comprising new genomic testing variables; and
predicting, using the trained machine learning model and new genomic testing variables, whether the new genomic workflow pipeline will be successfully completed within a first compute environment.

12. The system of claim 11, wherein the training datasets comprise, for each of a plurality of genomic tests, a corresponding batch size, sample size and queue size.

13. The system of claim 11, wherein the machine learning model comprises a supervised logistic regression model.

14. The system of claim 11, generating the prediction further comprising generating a user interface with a plurality of input elements that correspond to the new genomic testing variables.

15. The system of claim 14, wherein the input elements comprising sliders.

16. The system of claim 11, wherein the new genomic testing variables correspond to the plurality of training variables.

17. The system of claim 11, wherein the training variables comprising a corresponding compute environment.

18. The system of claim 17, wherein the training variables comprises an amount of memory and a number of central processing units, the generating the prediction further comprising a recommendation of a new compute environment for executing the new genomic workflow pipeline.

19. The system of claim 18, wherein the recommendation comprises a selection of one of a plurality of pre-configured cloud compute environments, the recommendation based at least in part on a cost of each of the pre-configured cloud compute environments.

20. A computer readable medium having instructions stored thereon that, when executed by one or more processors, cause the processors to predict genomic testing using machine learning, the predicting comprising:
receiving one or more training datasets of a genomic pipeline comprising a plurality of training variables for each of a plurality of genomic tests and corresponding results of each of the genomic tests;
training a machine learning model using the training datasets;
receiving a new genomic workflow pipeline comprising new genomic testing variables; and
predicting, using the trained machine learning model and new genomic testing variables, whether the new genomic workflow pipeline will be successfully completed within a first compute environment.
